(19) United States
(12) Patent Application Publication
(10) Pub. No.: US 2025/0366908 A1
(43) Pub. Date: Dec. 4, 2025

(54) METHODS AND TOOLS FOR MYOCARDIAL TISSUE

(71) Applicant: Mayo Foundation for Medical Education and Research, Rochester, MN (US)

(72) Inventors: Samuel J. Asirvatham, Rochester, MN (US); Freddy Del-Carpio Munoz, Rochester, MN (US); Ammar M. Killu, Rochester, MN (US); Jason A. Tri, Rochester, MN (US); Amir Lerman, Rochester, MN (US); Elad Maor, Tel Aviv (IL)

(21) Appl. No.: 18/873,471

(22) PCT Filed: Jun. 21, 2023
(86) PCT No.: PCT/US2023/025824

§ 371 Date: Dec. 10, 2024

Related U.S. Application Data
(60) Provisional application No. 63/354,494, filed on Jun. 22, 2022.

(51) Int. Cl. A61B 18/14; A61B 18/00; A61N 5/06

(52) U.S. Cl. CPC A61B 18/1492; A61B 18/1477; A61N 5/0601; A61B 2018/0022; A61B 2018/00351; A61B 2018/00577; A61B 2018/00642; A61B 2018/00839; A61B 2018/00875; A61B 2018/1435; A61B 2018/1467; A61N 2005/0612

(57) ABSTRACT

An electroporation catheter device comprising: a catheter shaft; and a helical anchor or needle member that is selectively extendable from a distal end of the catheter shaft, wherein the helical anchor member is configured to deliver pulsed-electric field non-thermal electroporation ablation energy to tissue of a heart. The device may be used in a method for treating ventricular fibrillation of a heart.

### CROSS-REFERENCE TO RELATED APPLICATIONS

This application claims the benefit of U.S. Provisional Application Ser. No. 63/354,494 filed Jun. 22, 2022. The disclosure of the prior application is considered part of (and is incorporated by reference in) the disclosure of this application.

### BACKGROUND

### 1. Technical Field

This document relates to methods, devices and systems for treating myocardial tissue. For example, this document relates to methods, devices and systems for delivering pulsed-electric field electroporation for ablation, with high tissue specificity for destruction while minimizing collateral damage to critical structures of the heart and extracardiac structures.

### 2. Background Information

Ventricular fibrillation (also referred to herein as “VF”) is a lethal rhythm that can result in sudden cardiac death (SCD). This is the number one cause of death—greater than all deaths from cancer in the United States combined. There is no cure for ventricular fibrillation that can lead to SCD—only treatments which are aimed at prevention of SCD such as drug therapy (which may be ineffective and fraught with side effects). ICD (“implantable cardiac defibrillator”) therapy is protective and could shock the patient back into normal rhythm, but also portends patients to ineffective shocks, inappropriate shocks, as well as post-traumatic stress disorder from receiving shock therapy. Radiofrequency (RF) ablation is limited in efficacy and issues with thermal ablation could lead to complications and unwanted tissue destruction. Although defibrillators, anti-arrhythmics, and other therapies provide an element of protection in select cases, sudden cardiac death remains a major worldwide health problem.

Ablation of deep interventricular septal substrate presents several challenges when employing conventional thermal energies such as radiofrequency ablation (RFA). Inadequate lesion depth is one major limitation despite multiple innovations aimed at enhancing mid-myocardial lesion formation. Furthermore, risks of thermal ablation in this region include coronary artery injury, atrioventricular conduction block, and thrombogenicity from heat-induced coagulum formation, steam-pops, and endocardial denudation.

Electroporation is a technique that uses very brief pulses of high voltage to introduce multiple nanopores within the cells' wall in a non-thermal manner (unlike RF), specifically within the lipid bilayer of the cell membranes as a result of the change in electrical field. Depending on the voltage and frequency of pulsations used, these pores can be reversible (i.e., increase the permeability of these cell to chemotherapeutic agents) and or irreversible (“IRE”; triggering cell death by the process of apoptosis or necrosis). Given the different composition of each cell-type membrane, electroporation can allow for a differential effect on different tissues.

### SUMMARY

This document describes methods, devices and systems for treating myocardial tissue. For example, this document describes methods, devices and systems for delivering pulsed-electric field electroporation for ablation, with high tissue specificity for destruction while minimizing collateral damage to critical structures of the heart and extracardiac structures.

In one aspect, this disclosure is directed to an electroporation catheter device that includes a catheter shaft and a helical anchor member that is selectively extendable from a distal end of the catheter shaft. The helical anchor member is configured to deliver pulsed-electric field non-thermal electroporation ablation energy to tissue of a heart.

Such an electroporation catheter device may optionally include one or more of the following features. The electroporation catheter device may also include a balloon member attached at a distal end portion of the catheter shaft. The electroporation catheter device may also include one or more electrodes on an outer surface of the balloon or in an interior of the balloon. The electroporation catheter may also include one or more straight needles that selectively extendable from the distal end of the catheter shaft and/or within an interior region defined by the helical anchor member. The electroporation catheter device may also include one or more electrodes on the catheter shaft. In some embodiments, the electroporation catheter may comprise an electrode cap to act as a return, sensing, pacing, or mapping electrode. The electroporation catheter device may also include a location sensor to assist in anatomical mapping. The catheter shaft or the helical anchor member may define a port for delivering a fluid. The catheter shaft or the helical anchor member may be configured to deliver photo-biomodulation light.

In another aspect, this disclosure is directed to a method for treating ventricular fibrillation of a heart. The method includes using one or more of the electroporation catheter devices defined herein to deliver pulsed-electric field non-thermal electroporation ablation energy to one or more target locations of the heart.

Such a method may optionally include one or more of the following features. The one or more target locations of the heart may include an endocardial space of the heart, the mid-myocardium of the heart, and an epicardial space of the heart. The one or more target locations of the heart may include a ventricular septum of the heart. The method may also include using two of the electroporation catheter devices and penetrating the ventricular septum with two of the helical anchor and/or needle members on a same side of the ventricular septum. In some embodiments, a first one of the helical anchor members functions as an anode and a second one of the helical anchor members functions as a cathode. The method may also include using two of the catheter devices and penetrating the ventricular septum with two of the helical anchor or needle members on opposite sides of the ventricular septum. In some embodiments, a first one of the helical anchor or needle members functions as an anode and a second one of the helical anchor members functions as a cathode.

Particular embodiments of the subject matter described in this document can be implemented to realize one or more of the following advantages: targeting of deep intramyocardial arrhythmic substrate; ablation without tissue heating in IRE mode only (RF via the device can still be used if desired); maintenance of cell architecture; Recording of intramyocardial signals (unipolar and bipolar depending on needle design); delivery of unipolar and bipolar electroporation/ablation; short ablation procedure times; catheter stability due to needle design; improved accuracy with clear demarcation line between ablated zone and healthy tissue; ability to delivery IRE, RF, microwave, cryo-ablation, or via photo-modulation; ability to delivery pulses from nanosecond to millisecond in duration; and ability to delivery fluid to the area of interest (e.g., drug delivery, calcium, saline, biologic, etc.). The methods and devices for electroporation can also limit damage to cardiac conduction tissue, coronary arteries and veins, phrenic nerve, pulmonary, bronchial, cardiac valves, cardiac ganglia, and normal cardiac muscle by selectively targeting tissues for ablation based on differences in tissue response. The systems and methods can provide superficial and/or deep myocardial ablations that are far reaching to accommodate variations in the shape of the ventricle and wide-areas of desired tissue effects. In some embodiments, a combination of two or more different types of ablation and/or electroporation energy can be delivered using the devices and methods described herein. For example, in some embodiments radiofrequency (RF) energy can be delivered concurrently or sequentially with pulses of direct current (DC) energy. Such delivery of multiple energy types can be leveraged, as described further below, to enhance the overall effects provided by the devices and methods described herein. In another aspect, a low voltage, reversible pulse or pulse train may be delivered to the target tissue prior to treatment to alter the impedance of the target tissue. The impedance of the target tissue can be sensed through the electrodes, needles, or helix prior to, during, and after ablation to use as an assessment of ablation. Alternatively, machine learning may be used to systematically alter the delivery settings to get the desired impedance change of the target tissue. The following method will increase the safety and efficacy of the procedure and the method may be done with any energy source (e.g., RF, microwave, PFA, cryo-ablation, ultrasound, biologics, etc.). Similarly, the systems and methods can provide these ablation lesions to occur in proximal and distal regions of the specialized conduction tissue of the heart (His-purkinje system), as well as distal or proximal only.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings and from the claims.

### DESCRIPTION OF DRAWINGS

FIG. 1 is a cross-sectional view of a human heart showing an example pulsed electric field electroporation procedure being administered to the ventricular septum of the heart.

FIG. 2 are radiographic images of the arrangement of FIG. 1.

FIG. 3 shows surface electrocardiogram (ECG) and intracardiac electrograms (EGM) before (“Baseline” graphs) and immediately after pulsed electric field delivery (“Post” graphs) across the interventricular septum.

FIG. 4 shows the progression over time of reversible atrioventricular (AV) conduction impairment following pulsed electric field delivery across the interventricular septum.

FIG. 5 shows magnetic resonance images of a portion of the heart taken prior to a pulsed electric field electroporation procedure, early after the pulsed electric field electroporation procedure, and later after the pulsed electric field electroporation procedure.

FIG. 6 is a graph that shows that the left ventricular (LV) diastolic mass calculated by volumetrics did not change significantly from baseline.

FIG. 7 is a graph that shows that the left ventricular ejection fraction decreased from baseline to early imaging but did not differ significantly from baseline by late.

FIG. 8 shows the histological characteristics of ablation lesions. Triphenyltetrazolium chloride-stained heart sliced in short-axis to show a near transmural pale lesion.

FIG. 9 is a cross-sectional view of a human heart showing another example pulsed electric field electroporation procedure being administered to the ventricular septum of the heart.

FIG. 10 shows an example low energy pulsed electric field electroporation procedure being administered using a single electroporation catheter.

FIG. 11 shows an example high energy pulsed electric field electroporation procedure being administered using two electroporation catheters.

FIG. 12 shows a distal end portion of another example electroporation catheter that can be used to perform a pulsed electric field electroporation procedure.

FIG. 13 shows a distal end portion of another example electroporation catheter that can be used to perform a pulsed electric field electroporation procedure.

FIG. 14 shows a distal end portion of another example electroporation catheter that can be used to perform a pulsed electric field electroporation procedure.

FIG. 15 shows a distal end portion of another example electroporation catheter that can be used to perform a pulsed electric field electroporation procedure.

Like reference numbers represent corresponding parts throughout.

### DETAILED DESCRIPTION

This document describes methods, devices and systems for treating myocardial tissue. For example, this document describes methods, devices and systems for delivering pulsed-electric field (PEF) electroporation for ablation, with high tissue specificity for destruction while minimizing collateral damage to critical structures of the heart and extracardiac structures.

In an overall aspect, the subject of the impedance modification as described in detail herein provides methods for increasing the efficacy and safety of ablation to tissue such as cardiac tissue. In some embodiments, the first step of such methods includes delivering low ablation threshold energy (e.g., RF, cryo, PEF, microwave, ultrasound, biologic, etc.) to modify the impedance of the target tissue (e.g., myocardial tissue) at the point of ablation. Following the modification of the target tissue, a baseline impedance measurement can be taken to provide a starting impedance in the impedance algorithms described herein. Such an ‘algorithm’ refers to the steps involved in making an assessment/evaluation for ablation completion or probability that a long-term lesion will form, limiting recurrence. Following tissue impedance change, the methods can include delivery of electroporation/ablation energy to form one or more lesions to the target tissue. During the delivery of electroporation/ablation energy, the impedance of the target tissue can be sensed in between each energy pulse via a multiplexing circuit to measure tissue impedance and signal potential. This tissue impedance can be tracked and used to determine and/or allow the energy settings (e.g., pulse width, pulse duration, current, voltage, etc.) to be altered, allowing the impedance to change to a desired target goal set in the ablation parameters. In some embodiments, ablation completion will be marked by a certain percentage change in the impedance of the target tissue and/or signal amplitude. The impedance change can vary depending on the tissue characteristics (e.g., atrial tissue, ventricular tissue, ventricular septum tissue, etc.). The final tissue impedance following ablation can be inputted into software allowing the calculation for the probability of chronic lesion formation. In addition, in some embodiments this algorithm can be inputted into a modeling software program demonstrating the electric field, lesion, and ablation parameters based off of individual anatomy.

In accordance with the methods, devices and systems for treating myocardial tissue described herein, bipolar irreversible PEF electroporation can be delivered to the interventricular septum to produce deep septal lesions. Multiple studies, as described herein, were performed by the inventors to develop and evaluate the efficacy, electrophysiologic, imaging, and histological characteristics of direct-current PEF delivered across the interventricular septum to produce deep septal lesions to treat ventricular arrhythmias, for example.

### Study #1

A first study evaluated effects of PEF when delivered across the interventricular septum of healthy canines in a bipolar fashion. Transient atrioventricular block occurred in most animals and bundle branch block persisted in some. Ventricular fibrillation did occur with microsecond PEF delivery. Early MRI showed edema and ventricular dysfunction which improved by late imaging. Lesions up to 10.9 mm were seen on histology.

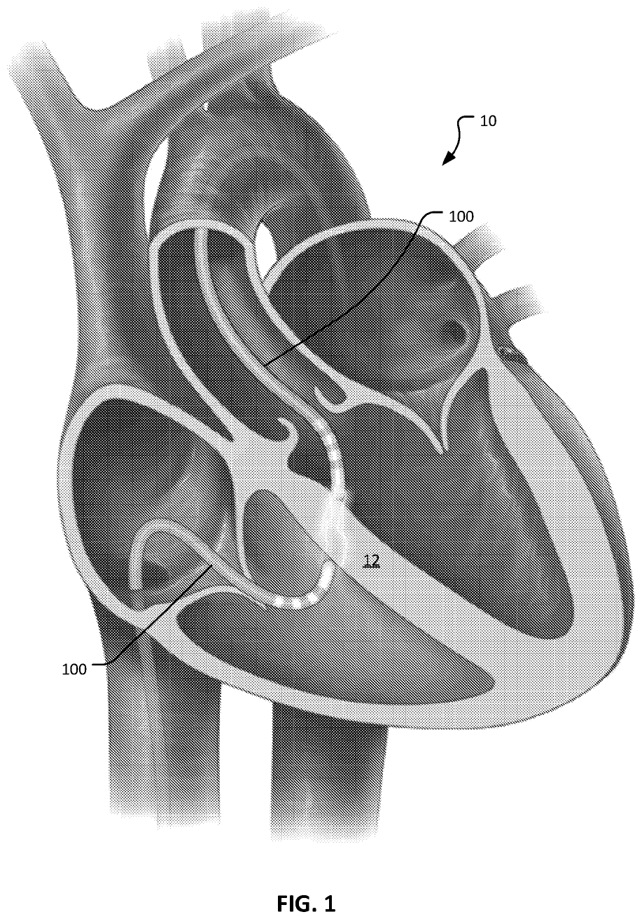

As depicted in FIG. 1, PEF was applied between identical solid-tip ablation catheters 100 positioned on either side of an interventricular septum 12 in a chronic canine model heart 10. Intracardiac and surface electrophysiologic data were recorded following delivery. Magnetic resonance imaging (MRI) was performed in 4 animals early (6±2 days) and late (30±2 days). After 4 weeks of survival, cardiac specimens were sent for histopathology.

Across the eight (8) canines in the study, PEF was delivered in 20 septal sites (45±17J/site). Transient complete atrioventricular block was seen in 5 animals (63%) after delivery at the anterobasal septum. Of these, bundle branch block persisted in 3 (38%). Ventricular fibrillation occurred during microsecond but not nanosecond PEF delivery. Two animals died acutely due to complications not directly related to PEF delivery. Early MRI showed prominent edema and significant left ventricular systolic dysfunction which improved on late imaging. At 4 weeks, 38 individual well-demarcated near transmural lesions were demonstrated by MRI and histopathology. Lesion depth measured by histology was 2.6±2.1 mm (maximum 10.9 mm).

Based on the outcomes of the study, bipolar irreversible electroporation of the interventricular septum 12 is feasible and can produce deep lesions. Ventricular stunning, myocardial edema, and conduction system injury may occur at least transiently.

Ablation of deep interventricular septal substrate presents several challenges when employing conventional thermal energies such as radiofrequency ablation (RFA). Inadequate lesion depth is one major limitation despite multiple innovations aimed at enhancing mid-myocardial lesion formation. Furthermore, risks of thermal ablation in this region include coronary artery injury, atrioventricular conduction block, and thrombogenicity from heat-induced coagulum formation, steam-pops, and endocardial denudation.

PEF is a non-thermal energy source induces cell death through a mechanism of irreversible electroporation—unstable pore formation in the cell membrane and intracellular organelles resulting in disruption of homeostasis and activation of the apoptotic cascade. Unlike thermal ablation, which results in nonspecific coagulative necrosis, the electric field intensity threshold to induce cell death differs across various cell types and appears to be relatively low for myocardium. This feature may portend a decreased risk of collateral injury to the coronary arteries and, perhaps, the proximal specialized conduction system.

Clinical trials utilizing PEF for cardiac applications are currently underway but are focusing primarily on atrial ablation. Ventricular PEF has been reported in a few preclinical studies using various delivery configurations and energy parameters. The ability to produce myocardial ablation at a given set of parameters while sparing other critical tissues makes PEF an attractive modality for treatment for treatment of deep septal substrate. As a proof-of-concept, this study aimed to evaluate the electrophysiologic, imaging, and histological characteristics of high-amplitude direct-current PEF delivered across the septum of healthy canines in a bipolar fashion from symmetrical catheters.

For the canine study, the right common femoral artery and vein were accessed via a cut-down approach. After access was obtained, intravenous heparin was bolused (100 IU/kg). Intracardiac echocardiography and fluoroscopy were used to guide catheter manipulation. Surface electrocardiogram (ECG) signals were processed with a band-pass filter of 0.05-100 Hz, and intracardiac electrogram (EGM) signals were filtered at 30-500 Hz bipolar and unipolar signals to 0.05-500 Hz. Unipolar EGMs used the Wilson's Central Terminal as reference. Intraprocedural pacing was performed using a four-channel Bloom Stimulator.

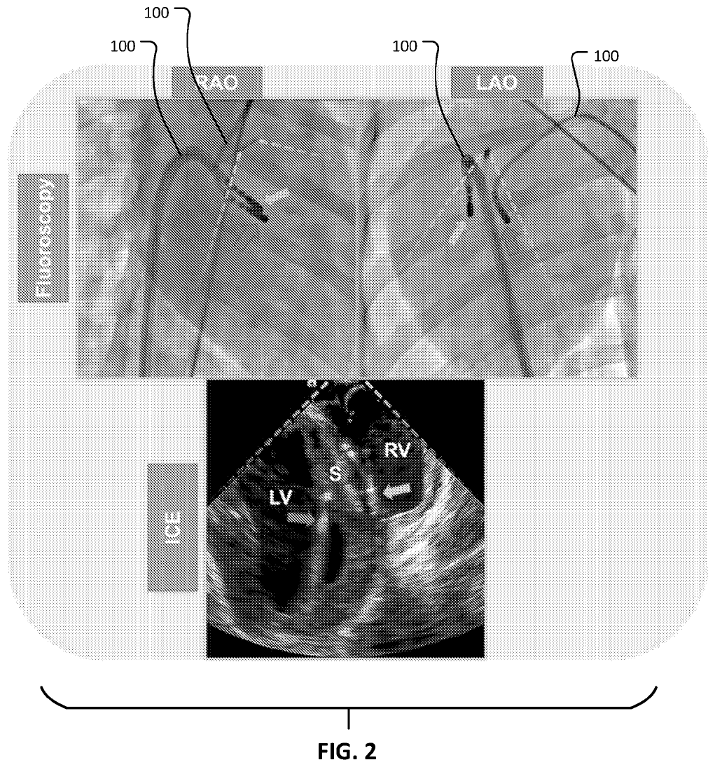

An 8.5 Fr medium curl steerable introducer was used to improve stability in the right ventricle (RV). The left ventricle (LV) was entered through a retrograde transaortic approach. Two solid-tip non-irrigated deflectable ablation catheters 100 (FIGS. 1 and 2) were positioned on both sides of the basal and mid interventricular septum 12.

Bipolar EGM signals were recorded at baseline from each catheter 100 and amplitudes were measured from peak-to-peak. Pacing thresholds were established at baseline by dialing down from maximum output (20 mA @ 0.5 ms). If local capture was intermittent or absent at maximum output, the procedure was repeated at 1.0 ms followed by 2.0 ms pulse widths. Coronary angiography was performed at baseline using selective engagement of the left main coronary artery using diagnostic angiography catheters and iohexol contrast (up to 100 mL). The right coronary artery was not engaged due to its small size in the canine and significant distance from the ablation target.

In the first two animals, microsecond duration direct-current pulses were delivered using the NanoKnife generator (AngioDynamics, Latham, NY) with gating to the R wave on the surface ECG using a cardiac trigger monitor. A stable monophasic square-wave pulse width (100 μs) was delivered with variation in the output (1000-1500V) and number of pulses (40-60) per site. In the following six animals, nanosecond duration direct-current pulses were delivered using the CellFX generator (Pulse Biosciences, Hayward, CA) at a stable pulse width (300 ns) with waveform, number of pulses, amplitude, and pulse frequency considered proprietary data at the request of the manufacturer. Nanosecond duration pulses were delivered at a fixed frequency. Total joule energy delivered was calculated using generator stored logs of actual current delivered, voltage, pulse duration, and number of pulses. PEF was delivered through the basal and mid interventricular septum 12 in a bipolar configuration from catheter 100 electrodes on one side of the septum 12 (anode) to those on the other side of the septum 12 (cathode) in all animals. While maintaining stable catheter 100 positions, signals were recorded and pacing thresholds were reestablished 5 minutes following PEF delivery.

Sustained ventricular arrhythmias were defined as ventricular tachycardia and ventricular fibrillation requiring electrical cardioversion/defibrillation for hemodynamic instability or lasting greater than 30 seconds without spontaneous termination. Atrioventricular (AV) block was defined as complete AV dissociation consisting of at least five non-conducted P waves.

RFA control lesions at non-septal sites were performed for comparison purposes in some animals. Sedation was weaned, post-operative antibiotics, and analgesia were provided for 5 days following the procedure. Animals were examined for complications daily for 1 week then 3 times weekly throughout the survival period.

Cardiac magnetic resonance imaging (MRI) was performed in vivo in 4 animals within 7 days (early) post-procedure and again immediately prior to the end study at approximately 30 days (late). During MRI, animals underwent the same anesthesia protocol as outlined above during the MRI scan. Cardiac and respiratory gating were performed. Balanced steady-state gradient echo and T2-weighted triple inversion recovery sequences were obtained. Intravenous gadodiamide contrast (0.2 mmol/kg) was infused to facilitate perfusion imaging and late gadolinium enhancement (LGE) sequences obtained 10-25 minutes following infusion.

Volumetric cardiac measurements were performed by importing images into medical image post-processing software. Short axis planes were used to semi-automatically draw regions of interest that were manually adjusted according to methods previously validated in canine studies. The endocardial and epicardial borders of the LV were included from apex to the level where greater than 25% of the annulus included basal ventricular myocardium. The RV endocardial border was drawn to the tricuspid and pulmonic annuli. Papillary muscles were included within both RV and LV volumes for consistency. Volumes were obtained at end-systole and end-diastole to allow software calculation of RV and LV ejection fraction (EF) as well as LV mass at end-diastole.

MRI data was compared to baseline MRIs performed for a previous study at this institution utilizing the same imaging protocol from four canines matched by age, weight, and gender.

Cardiectomy was performed during necropsy. The hearts 10 were incubated in a 3% solution of triphenyltetrazolium (TTC) to highlight regions of gross ablation. All hearts 10 were examined and sliced in 3 roughly equal thickness short-axis segments (apex, middle, and base). The interventricular septum 12 and other sites with obvious myocardial ablation were further sectioned. Specimens were photographed prior to formalin fixation. Histology sections were created with hematoxylin and eosin as well as Masson trichrome staining. Slides were qualitatively evaluated blindly by a single independent board-certified pathologist. The dimensions of the lesions were obtained by measuring the maximum depth perpendicular to endocardium reported in millimeters (mm) on the digital images. Lesion volume (V) was estimated using a half ellipsoid volume formula incorporating lesion width and depth

$$( V = \frac{2}{3} π \cdot {( \frac{1}{2} width )}^{2} \cdot depth ) .$$

Results were presented using descriptive statistics. Normally distributed continuous variables are reported as mean±standard deviation, nonnormally distributed variables as median [interquartile range], and categorical variables as percentages, unless otherwise specified. Continuous variables were compared using paired t-tests to compare values before and after delivery in the same animal. Comparison to matched baseline MRI values was performed using an unpaired t-test. For these statistical comparisons, 95% confidence intervals (95% CI) were calculated. Statistical analysis was performed.

### Results

### Acute Energy Delivery

As summarized in Table 1 below, in eight canines with a mean weight 35.8±3.8 kg, PEF was delivered in a total of 20 individual sites (1-5 sites/animal) across the basal to mid interventricular septum.

TABLE 1

| | | | | Total | | | |
|---|---|---|---|---|---|---|---|
| | | | | Energy | Sustained | Atrio- | Initial |
| | Weight | Pulse | Individual | Delivered | Ventricular | \|ventricular | Study |
| Animal | (kg) | Generator | Sites | (J) | Arrhythmia | Block | Survival |
| #1 | 36.1 | NanoKnife | 1 | 75.0 | 1 | 0 | 0 |
| #2 | 34 | NanoKnife | 3 | 189.3 | 0 | 1 | 1 |
| #3 | 44.1 | CellFx | 3 | 117.4 | 0 | 0 | 1 |
| #4 | 32 | CellFx | 4 | 107.4 | 0 | 0 | 1 |
| #5 | 37 | CellFx | 5 | 275.4 | 1* | 1 | 0 |
| #6 | 37.1 | CallFx | 3 | 100.6 | 0 | 1 | 1 |
| #7 | 31 | CellFx | 5 | 120.2 | 0 | 1 | 1 |
| #8 | 35.2 | CellFx | 3 | 135.0 | 0 | 1 | 1 |

*Intractable ventricular fibrillation occurred during a radiofrequency control lesion.

A total of 140±60 J of energy was delivered per animal with an average of 45±17 J delivered per site. Of these, 4 sites (66±5 J per site) were treated with the NanoKnife generator with 100 μs pulse duration and 16 sites (37±12 J per site) were treated with the CellFx generator with a 300 ns pulse duration. Microbubbles were seen on intracardiac echocardiography during microsecond but not nanosecond delivery. Bipolar microsecond PEF delivery resulted in obvious skeletal muscle stimulation and intravenous paralytic was administered to improve catheter stability and allow completion of the protocol. Nanosecond delivery did not result in noticeable extracardiac muscle stimulation in the absence of paralytic.

### Acute Post-Delivery Electrograms and Arrhythmia

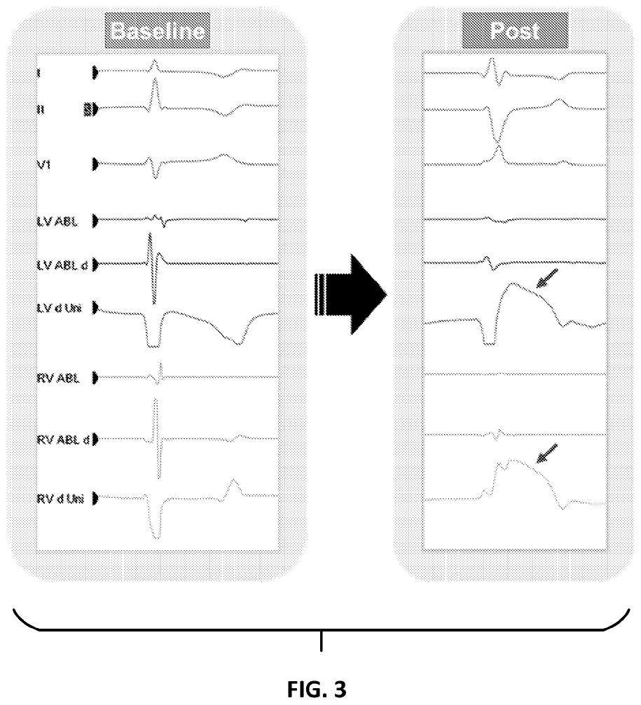

Local bipolar ventricular EGM amplitudes were 4.1±2.7 mV before PEF delivery and 2.1±1.6 mV after delivery (p<0.001). Local myocardial capture thresholds increased from 5.3±5.1 mA @ 0.5 ms to 17.0±6.3 mA @ 0.5 ms immediately following delivery (p<0.001) and complete loss of capture was demonstrated at 11 sites (61%). Prominent post-systolic elevation (injury current) was reliably seen on unipolar EGMs after delivery at all sites (see FIG. 3).

FIG. 3 shows surface electrocardiogram (ECG) and intracardiac electrograms (EGM) before (“Baseline” graphs) and immediately after pulsed electric field delivery (“Post” graphs) across the interventricular septum. Following energy delivery, a right bundle branch block pattern is seen on surface ECG leads I, II, and V1. Local bipolar EGM amplitudes are greatly diminished from Baseline as recorded on ablation (ABL) catheters on both sides of the septum. Prominent post-systolic elevation (injury current) was seen on unipolar (Uni) recordings after delivery (see arrows in “Post” graphs).

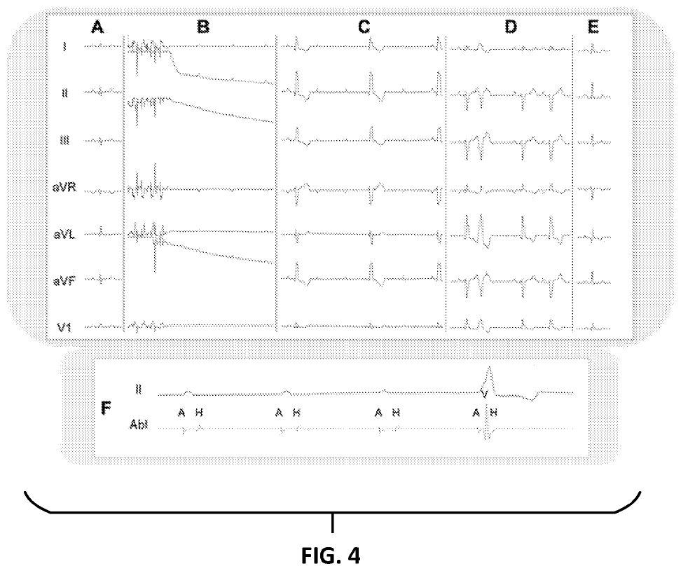

Following PEF, changes in AV conduction and surface ECG morphology were seen in all eight animals at least transiently. In five animals, temporary complete AV block was seen lasting between 0:12 to 61:58 minutes before recovering one-to-one AV conduction. One animal had hemodynamically stable persistent AV block through the duration of the acute study after 94:21 minutes (#6) which persisted after weaning sedation with no apparent detriment to the animal once awake. Duration of AV block appeared to be site and dose dependent, only occurring with delivery at the basal anteroseptum; with single test pulses resulting in very brief AV block and full delivery doses persisting much longer. The site of AV block was infrahisian in those where a His potential could be recorded (see FIG. 4). Both left and right bundle branch block patterns were seen at least transiently.

FIG. 4 shows the progression over time of reversible atrioventricular (AV) conduction impairment following pulsed electric field delivery across the interventricular septum. In animal #7, the surface electrocardiogram was recorded at baseline (Panel A). Artifact from the end of the energy delivery sequence is followed by complete AV block (Panel B) with a ventricular escape coming in 7 seconds later (Panel C). Conduction recovered after 2:06 minutes with a right bundle branch block morphology (Panel D). By 60 minutes, conduction had normalized with QRS and PR intervals similar to baseline (Panel E). Electrograms recorded at slower paper speed (100 mm/sec) from the ablation catheter (Abl) during AV dissociation suggested infrahisian block (Panel F).

Brief (<10 second) accelerated ventricular and junctional rhythms were seen following delivery with microsecond and nanosecond PEF. No animals developed sustained ventricular arrhythmias with nanosecond PEF delivery. Animal #1 developed ventricular fibrillation during appropriately synchronized ECG-gated microsecond PEF delivery. The animal was successfully defibrillated externally without incident. Later, the animal developed refractory hemodynamic instability requiring the study to be terminated. Coronary angiography showed widely patent epicardial coronary arteries. Necropsy demonstrated mediastinal hemorrhage secondary to an aortic perforation likely related to retrograde transaortic catheter manipulation. No myocardial ablation was evident acutely.

Another animal (animal #5) developed intractable ventricular fibrillation during an RFA control lesion performed more than 15 minutes after completion of nanosecond PEF delivery. Coronary arteries were widely patent by angiography in this animal. The RFA lesion at the apex was within a region of prominent endocardial Purkinje fibers on necropsy.

### Magnetic Resonance Imaging

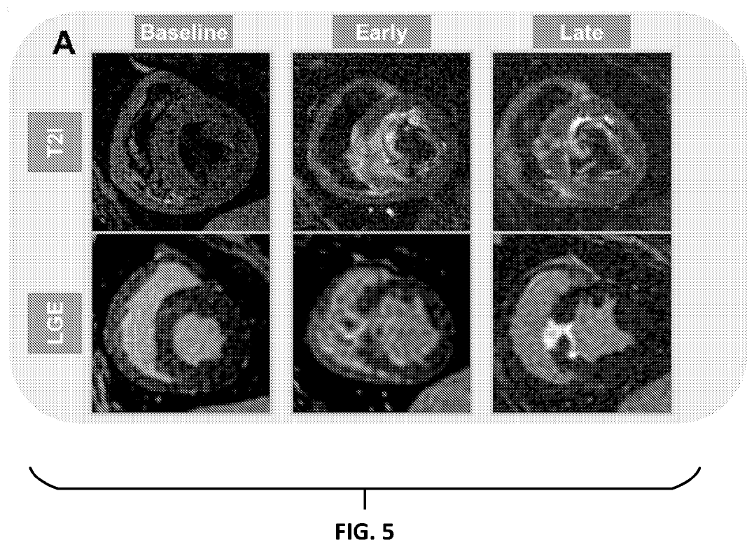

In four animals (#2, #4, #7, and #8), gadolinium enhanced MRI was performed with “Early” imaging at 6±2 days and with “Late” imaging at 30±2 days (see FIG. 5). Magnetic resonance imaging results following energy delivery. Short-axis slices of a single representative animal (#8) showing at “Early” (7 days) increased septal thickness, patchy bright T2-weighted signal, and poorly demarcated patchy late gadolinium enhancement (LGE). “Late” imaging (30 days) demonstrated regression of septal thickening, less T2-weighted signal, and well demarcated transmural LGE in a similar distribution.

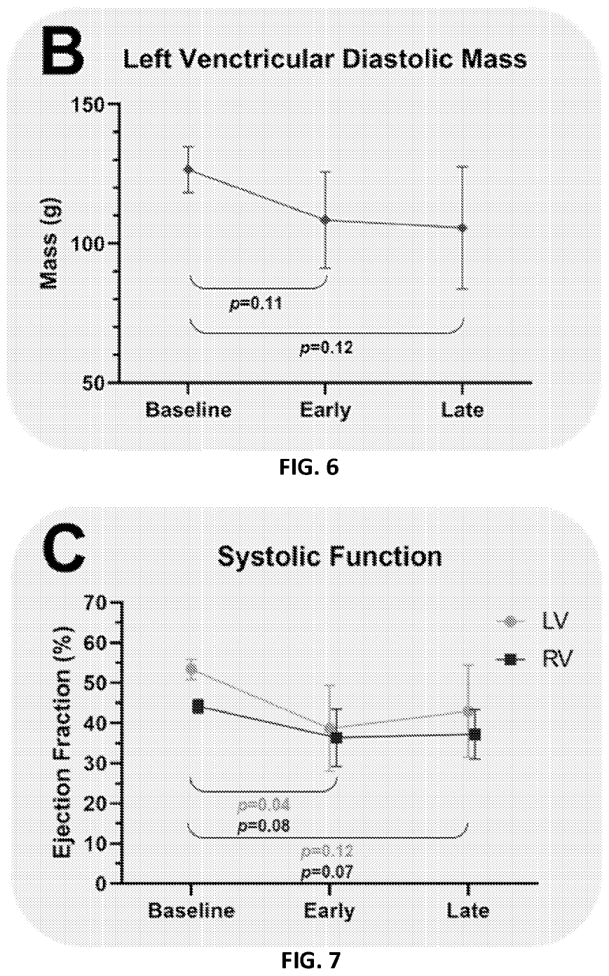

FIG. 6 shows that the left ventricular (LV) diastolic mass calculated by volumetrics did not change significantly from baseline.

FIG. 7 shows that the left ventricular ejection fraction decreased from baseline to early imaging but did not differ significantly from baseline by late. Right ventricular ejection fraction did not differ significantly from baseline to early imaging or late imaging.

Within the basal and mid interventricular septum, early findings consistently showed increased septal thickness, patchy bright T2-weighted signal, and poorly demarcated patchy LGE. Late imaging demonstrated regression of septal thickening, less T2-weighted signal, and well demarcated LGE in a similar distribution compared to abnormalities on early imaging. Transmural LGE was present in one animal (#8) with late imaging. Compared to matched baseline controls with a volumetrically calculated diastolic LV mass of 126.6±8.3 g, mass did not differ significantly on early imaging (−18.1 g, 95% CI: −41.6 to 5.4 g; p=0.11) or late imaging (−21.0 g, 95% CI: −41.6 to 5.4 g; p=0.12). LV ejection fraction (EF) decreased from baseline to early imaging from 53.4±2.5% to 38.7±10.7% (−14.7%, 95% CI: −28.2 to −1.3%; p=0.04) but did not differ significantly from baseline to late imaging (−10.5%, 95% CI: −24.8 to 3.9%; p=0.12). RV EF did not differ significantly from baseline of 44.2±1.7% to early imaging (−7.8%, 95% CI: −16.8 to 1.1%; p=0.08) or late imaging (−7.0%, 95% CI: −14.9 to 0.9%; p=0.07). One animal (#8) had substantial mass like septal swelling, septal akinesis, and biventricular systolic dysfunction (LV EF 23.2% and RV EF 28.6%) on early imaging which recovered partially (LV EF 46.9% and RV EF 38.2%) on late imaging.

### End Study and Lesion Characteristics

All six animals (100%) surviving the acute study also survived through end study at 30±2 days without interim complications. New right bundle branch block with 1:1 AV conduction was present at end study in three animals (animals #6 and #8) with AV conduction on ECG approximating baseline in the rest. Left coronary angiography did not show any epicardial coronary stenotic or occlusive lesions. Gross necropsy specimens showed a total of 38 lesions that were sectioned and sent for histopathology (see Table 2).

TABLE 2

| | | Mean | | | | | |
|---|---|---|---|---|---|---|---|
| | | Energy | | Maximum | Mean | Maximum | Mean |
| | | Delivered | Individual | Lesion | Lesion | Lesion | Lesion |
| | Pulse | Per Site | Histologic | Depth | Depth | Volume | Volume |
| Animal | Duration | (J) | Lesions | (mm) | (mm) | (mm³) | (mm³) |
| #1 | Microsecond | 75.0 | NA | NA | NA | NA | NA |
| | (100 us) | | | | | | |
| #2 | Microsecond | 63.1 | 5 | 3.0 | 1.7 ± 0.8 | 54 | 16 ± 22 |
| | (100 us) | | | | | | |
| | Nanosecond | 39.1 | 5 | 3.6 | 1.7 ± 1.1 | 59 | 15 ± 25 |
| | (300 ns) | | | | | | |
| #4 | Nanosecond | 26.9 | 7 | 4.8 | 3.1 ± 1.4 | 90 | 42 ± 32 |
| | (300 ns) | | | | | | |
| #5 | Nanosecond | 55.1 | NA | NA | NA | NA | NA |
| | (300 ns) | | | | | | |
| #6 | Nanosecond | 33.5 | 5 | 5.3 | 2.7 ± 1.9 | 100 | 45 ± 44 |
| | (300 ns) | | | | | | |
| #7 | Nanosecond | 24.0 | 10 | 2.5 | 1.6 ± 1.0 | 128 | 20 ± 39 |
| | (300 ns) | | | | | | |
| #8 | Nanosecond | 45.0 | 4 | 10.9* | 6.4 ± 3.3 | 206 | 108 ± 71 |
| | (300 ns) | | | | | | |

NA = Not applicable due to acute animal demise;
N = Not present;
P = Preserved;
I = Injured
*Near transmural lesion (contiguous across septum)

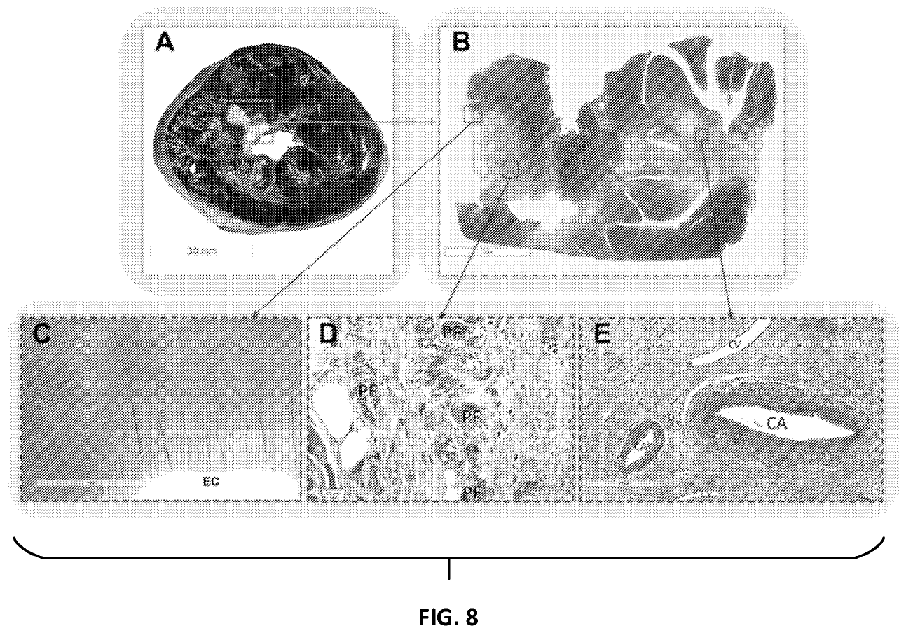

By histology, ablation was characterized as a well-demarcated absence or paucity of viable myocardial cells within residual homogeneous collagenous replacement adjacent to normal healthy myocardium. Superficial to ablation, the endocardial layer was preserved in 21/38 (55%) and adherent thrombus was not present in any sample. As measured by histopathology, mean lesion depth was 2.6±2.1 mm with a maximum depth of 10.9 mm that was contiguous across the interventricular septum in one animal (#8). Mean lesion volume was 36±46 mm³ with a maximum lesion volume of 206 mm³. Purkinje fiber tissue and intramyocardial coronary vessels were seen within 29/38 (76%) and 31/38 (82%), respectively, all with preserved architecture, intact nuclei, and no luminal stenosis or occlusion in observed samples (see FIG. 8).

FIG. 8 shows the histological characteristics of ablation lesions. Triphenyltetrazolium chloride-stained heart sliced in short-axis to show a near transmural pale lesion (Panel A). Masson trichrome staining of histology section showing healthy myocardium (pink) adjacent to myocardial ablation and connective tissue (blue) which appears contiguous across the septum (Panel B). Sharply demarcated myocardial ablation is noted with homogeneous residual extracellular connective tissue and collagen replacement with a normal endocardial (EC) lining overlying (Panel C). Specialized conduction tissue and Purkinje Fibers (PF) are seen with preserved ultrastructure and intact nuclei within a myocardial ablation lesion (Panel D). Microscopic intramural coronary arterioles (CA) and coronary venules (CV) have preserved architecture and no evidence of luminal stenosis or occlusion within myocardial ablation lesion (Panel E).

### Discussion

Using commercially available ablation catheters, this study described above examined the electrophysiologic, imaging, and histologic features of bipolar pulsed electric fields delivered across the interventricular septum.

Some of the significant findings from the study include:
1. Deep myocardial ablation, including near transmural lesions, is feasible using bipolar PEF across the interventricular septum.
2. The infrahisian conduction system can be affected at least transiently by PEF delivery across the basal septum.
3. Early MRI imaging after ventricular PEF shows prominent edema and systolic dysfunction preceding well-defined ablation in a similar distribution.
4. PEF ablation lesions contained normal intramural coronary vessels, viable conduction system tissue, and preserved endocardium.

### Lesion Formation

With the growing interest in PEF for atrial ablation, investigating the use of this energy modality in ventricular ablation is a logical next endeavor. The speed of delivery and perceived safety relative to RFA have fueled early preclinical investigation and even a case report of RV outflow tract ablation for ventricular ectopy in a human. Indeed, this study confirmed deep myocardial ablation achieved with bipolar PEF delivery across the interventricular septum. These ablation lesions occurred at sites where acutely reduced bipolar EGM amplitude, increased pacing thresholds, and post-systolic elevation of the unipolar EGM (injury current) were recorded. A maximum lesion depth of over 10 mm was present and lesions were nearly transmural and contiguous across the septum.

Previous reports have described similar ventricular lesion depths with PEF delivered by various configurations. With monopolar delivery from one catheter to a reference patch, ventricular lesions over 9 mm in depth have been described in a dose dependent fashion. Similarly, bipolar delivery using a biphasic waveform delivered from a single lattice-tip catheter has demonstrated lesion depths of up to 9 mm. Another study used alternating current did use a reference electrode in the right ventricle while delivering pulsed fields from a catheter in contact with the left ventricular side of the septum—modest lesion depths of under 6 mm were produced. These prior studies did not have symmetrical catheter electrodes directly in contact with both sides of the septal myocardium. This configuration offers the benefit of concentrating energy across the myocardium and inducing electroporation near electrodes on both sides of the septum.

The use of bipolar transseptal PEF for myocardial ablation offers promise as a modality to treat deep septal substrate for ventricular arrhythmias. In addition, as an alternative to alcohol septal ablation or surgery, this technique could be utilized to reduce septal mass for dynamic outflow tract obstruction as has been reported with RFA in small human series with both endocardial and percutaneous intramyocardial needle delivery methods. The configuration used in this study could also potentially be adapted for endocardial-epicardial bipolar ablation to treat mid-myocardial substrate in the ventricular free wall.

### Effect on Atrioventricular Conduction

Immediately after PEF delivery, infrahisian conduction abnormalities were seen in all animals consisting of either left bundle branch block, right bundle branch block, fascicular block, or complete AV block depending on the target location and energy dose delivered. Specifically, delivery across the basal anteroseptum appeared to be associated with the highest risk of infrahisian complete AV block. Although 1:1 AV conduction eventually returned in all animals, right bundle branch block persisted in three animals. Still, on histology, viable conduction system tissue and Purkinje fibers were present to some capacity within ablated myocardium in a majority. The mechanism by which the reversible conduction impairment occurs may include reversible electroporation, thermal injury, compression by edema, or mechanical injury through catheter manipulation.

For ventricular PEF, conduction system injury has disadvantageous but also potentially advantageous aspects. On one hand, the specialized cardiac conduction appears to vulnerable to irreversible electroporation and care must be taken to avoid these structures at doses intended to ablate myocardium. On the other hand, a transient dose-dependent effect has been reported by this group as well as others. This offers an opportunity to allow mapping of the conduction system using low dose PEF through the theoretical process of reversible electroporation. This could be utilized in a manner similar cryomapping for interrogation in proximity to or for arrhythmias arising from the conduction system (fascicular ventricular tachycardia, for example) prior to delivering an ablative dose.

Although acute Purkinje fiber dysfunction has been described previously following PEF with different parameters to this study, this may be a reversible phenomenon and, in keeping with the results of this study, clear irreversible Purkinje fiber ablation has not been confirmed by chronic histology.

### Imaging Characteristics

Serial in vivo cardiac MRI early and late provided important data that have not been previously described in myocardial PEF applications. Rather extensive T2 signaling and thickening of the septum was seen early after PEF delivery suggestive of a phase of prominent myocardial edema prior to cell death and fibrosis (as evidenced by well-defined LGE) on late imaging. Swelling has been well described at a cellular level where organelle and cell membrane pores result in osmotic shifts—drawing water into the cell. Eventually, some of these cells undergo cell death via the process of irreversible electroporation. These cellular mechanisms could explain the macroscopic findings seen on imaging. Although not seen during this series, one could anticipate that extensive edema resulting from PEF delivered in the outflow tract septum could place the subject at risk hemodynamically significant outflow tract obstruction at least transiently with clinical applications similar to what has been seen in a report with extensive RFA of the septum.

In addition to edema, significant global myocardial dysfunction was seen both subjectively and by volumetric measurements of biventricular EF on early imaging. Although there was not a statistically significant difference from baseline-matched controls, late imaging did also demonstrate lower numerical EF values with regional wall motion abnormalities primarily confined to areas of ablation. PEF delivery was likely more aggressive in this study than what would be required in clinical applications. Yet, there does appear to be some reversible myocardial stunning present by imaging within 7 days.

### Nanosecond as Opposed to Microsecond Energy Delivery

Although the sample sizes were too small to make quantitative comparisons, nanosecond duration PEF was not associated with any noticeable skeletal muscle contraction. Microsecond energy delivery did result in visible diaphragmatic and thoracic skeletal muscle contraction that, although mild, could result in catheter instability and provide substantial discomfort in a patient who has not received neuromuscular blockade and is not under general anesthesia. This observation has been reported previously with monophasic microsecond duration pulses in other preclinical and clinical studies with bipolar and biphasic configurations used as an attempt to mitigate the issue. Nanosecond pulse widths provide another strategy to reduce or eliminate extracardiac skeletal muscle stimulation.

In this study, sustained ventricular fibrillation requiring defibrillation was seen during microsecond but not nanosecond PEF. Although microsecond duration PEF delivered within the relative ventricular refractory period are known to result in ventricular fibrillation, this occurred despite adequate ECG-gating that has not been reported previously. There was no evidence of ischemia by ECG; but this animal was also later found to have an aortic perforation and it is possible that myocardial hypoperfusion may have contributed.

With the high-energy doses delivered for the purposes of ventricular ablation in this study, substantial microbubble formation was noted on intracardiac echocardiography during microsecond delivery but not nanosecond delivery. One widely held hypothesis is that these bubbles may be a result of current delivery inducing an electrolytic reaction releasing hydrogen and oxygen gas from water content in blood and tissue.

### Sparing of Coronaries and Endocardium

Similar to prior reports, this study showed no ECG evidence of myocardial ischemia and patent epicardial coronary arteries acutely. By histology, small intramural coronary vessels within ventricular PEF ablation lesions were preserved. Interestingly, the ventricular endocardial lining was also spared overlying a large proportion of ablation lesions with no adherent thrombus. This has also been reported by another study of atrial PEF. When compared to RFA ablation in the ventricle that is known to denude the endocardial lining acutely and induce coagulum through high temperatures, PEF could potentially reduce the subacute prothrombotic risk associated with ventricular ablation.

### Limitations

This preclinical investigation was performed using healthy canines. The doses used for PEF were exploratory and used to establish proof-of-concept. The study used commercially available non-irrigated ablation catheters designed for the purpose of RFA and not for tolerating high voltage DC pulses. Purpose designed catheters may provide more consistent and reproducible PEF delivery.

### Conclusions

Pulsed electric fields delivered across the interventricular septum in a bipolar configuration produced well-demarcated, deep, near transmural myocardial ablation lesions. This modality offers promise for ventricular ablation in clinical settings as an alternative to RFA. Prominent myocardial edema and ventricular stunning were evident on early post-procedural imaging which did improve at 4 weeks. Infrahisian impairment did occur, at least transiently, and care should be taken with PEF close to the specialized conduction system.

### Study #2

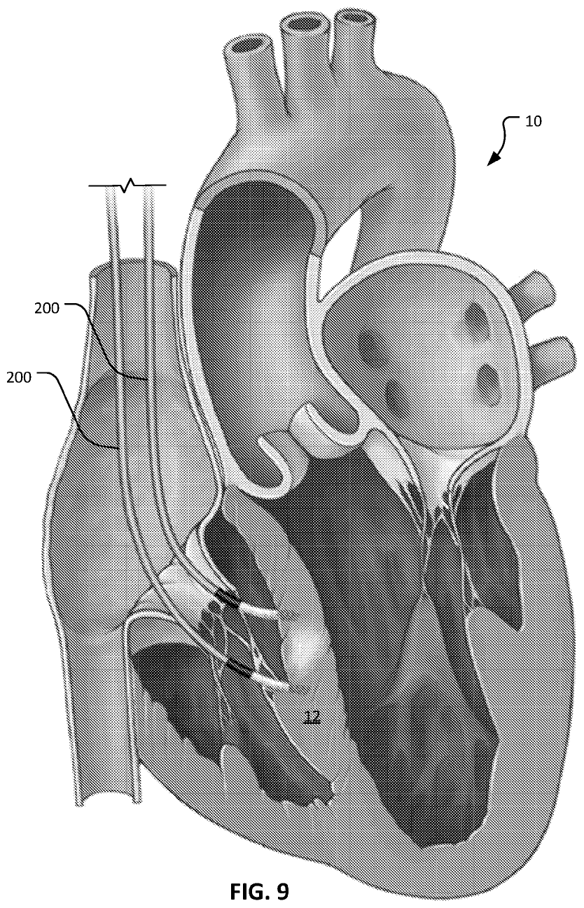

The inventors also performed a second study regarding the delivery of ventricular nanosecond pulsed electric field using active fixation leads. For example, the study tested PEF delivery using screw-in electroporation delivery leads (e.g., as depicted in FIGS. 9-11).

### Methods

In five canine models, nanosecond PEF (pulse width 300 ns) was delivered/applied across the right ventricular (RV) septum using a single lead bipolar configuration (n=2) and between two leads (n=3). Electrograms (EGMs) were recorded prior to, immediately post, and 5 minutes after PEF. Cardiac magnetic resonance imaging (cMRI) and histopathology were performed at 2 weeks and 1 month.

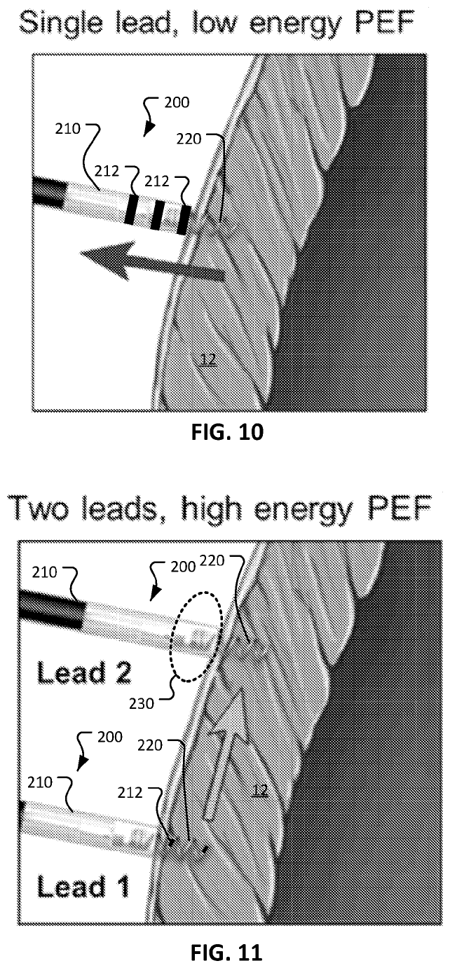

A single transvenous electroporation deliver lead was used in the first two experiments. The lead was guided towards the right ventricular (RV) septum using stylets and screwed into place as depicted in FIG. 10. PEF was performed with a helix-to-ring configuration. Pre-PEF, immediate post-PEF, and 5-minute post-PEF bipolar (helix-to-ring) as well as unipolar helix and ring EGMs were recorded. Other EGM changes including ST elevations in the unipolar electrograms were also noted. Pre- and 5-minute post-PEF pacing thresholds (at 0.5 ms) were obtained. To maximize animal use efficiency, multiple locations along the RV septum were attempted in each experiment. The electroporation lead was removed from the body following completion of PEF delivery. In addition, one representative RFA lesion was performed in the left ventricular (LV) free wall with the Thermocool® ablation catheter (Biosense Webster, Irvine, CA).

Because energy delivery was limited by the presence of arcing within a single electroporation lead, three additional experiments were performed utilizing two electroporation leads as depicted in FIGS. 9 and 11. Given the need for maneuverability, electroporation leads along with deflectable sheaths (C304 Select Site; Medtronic, Minneapolis, MN) were used. In each experiment, the electroporation leads were guided and fixated onto the RV septum at two distinct locations guided by fluoroscopy and impedance-tracking using an electroanatomical mapping system (Carto, Biosense Webster, Irvine, CA). PEF was delivered across the two electroporation leads: in two experiments, a helix-to-helix configuration was performed; in one experiment, a helix (of one lead) to ring (of the other lead) configuration was used due to high impedance errors with the helix-to-helix configuration. Pre-PEF, immediate post-PEF, and 5-minute post-PEF bipolar as well as unipolar helix and ring EGMs were recorded for each lead. Both electroporation leads were removed from the body following PEF delivery.

Voltage maps were created using Carto; both multielectrode (Penta-Ray; Biosense Webster, Irvine, CA) and single point catheters (Thermocool®; Biosense Webster, Irvine, CA) were used for mapping. For all experiments, RV maps were obtained at baseline, immediately post-PEF, and at end-study.

Cardiac magnetic resonance imaging (cMRI) was performed at two weeks and one month post-treatment. A 1.5T MRI scanner was used. Gadolinium was given for contrast enhancement; short and long axis images were also obtained 8-10 minutes post gadolinium administration to assess for myocardial delayed enhancement.

The canine hearts were inspected following end-study sacrifice, with particular attention paid towards the ventricular septum. Gross lesions were dissected and immersed in 10% neutral buffered formalin; when no apparent lesions were observed, normal-looking septal tissue samples were also sent to assess for microscopic pathology. Sections of 4 μm thickness were obtained; hematoxylin and eosin (H&E), periodic acid Schiff (PAS), and Masson's trichrome (MT) stains were applied where applicable. The slides were evaluated by a single board-certified pathologist, who was not blinded to the treatments performed.

Helix-to-helix PEF was performed in Studies 3 and 4, whereas helix-to-ring PEF was delivered in Study 5. Energy delivered ranged from 56.3-144.9J. Marked decreases in bipolar EGM amplitudes were seen post-PEF; most of these changes persisted past 5 minutes. Furthermore, prominent persistent ST elevations were seen in helix and ring unipolar EGMs in both studies as well as in the bipolar EGMs for Study 4.

Electroanatomic mapping immediately post-PEF did not show changes from baseline in Study 3; however, the end-study map showed decreased voltages in the mid and apical RV septum. Like Study 3, Study 4 did not show any voltage changes from baseline, but low voltage signals were seen on the RV septum 30 days post ablation. In Study 5, no abnormalities were noted on immediate post-PEF and end-study voltage mapping.

### Results

Minimal extracardiac stimulation with nanosecond PEF was seen across all experiments. Frequent ventricular ectopy occurred during energy delivery that terminated immediately post-treatment. No sustained ventricular tachycardia/fibrillation following PEF was noted. All canines survived to the duration of the end-study.

Nanosecond PEF was attempted in three and two locations for Study 1 (single electroporation lead) and Study 2 (two electroporation leads) respectively. Energy delivered ranged from 0.64-7.28J. Arcing on internal diagnostics was noted in two locations for Study 1, whereas elevated impedance was seen in one location for Study 2. Significant ST elevations were seen immediately post-PEF on unipolar helix and ring EGMs; these tended to decrease and normalize after 5 minutes. Small but significant decreases in bipolar EGM amplitudes were observed for Study 1. Due to technical issues with the recording system, EGMs for Study 2 were unavailable for review.

Immediate and end-study post-PEF electroanatomic maps did not reveal evidence of decreased voltage especially in the septal regions (corresponding with PEF delivery locations).

Nanosecond PEF induced minimal extracardiac stimulation and frequent ventricular ectopy that terminated post-treatment; no canines died with PEF delivery. With one lead (e.g., see FIG. 10), energy delivery ranged from 0.64-7.28J. Transient ST elevations were seen post-PEF. No myocardial delayed enhancement (MDE) was seen on cMRI. No lesions were noted on the RV septum at autopsy. With two leads (e.g., see FIGS. 9 and 11), energy delivery ranged from 56.3-144.9J. Persistent ST elevations and marked EGM amplitude decreases developed post-PEF. MDE was seen along the septum 2 weeks and 1 month post-PEF. There were discrete fibrotic lesions along the septum; pathology revealed dense connective tissue with <5% residual cardiomyocytes.

No MDE was seen in Studies 1 and 2 on both 2-week and 1-month cMRI scans. On gross inspection, no clear lesions were observed along the RV septum. Pathology examination of septal tissue revealed normal endocardium and myocardium.

No clear MDE was seen for Study 3 apart from a small focus in the RV apex of unclear etiology. However, on gross inspection, there were two distinct transmural lesions along the RV, corresponding to locations where the electroporation leads were placed and PEF delivered. Pathology revealed healed transmural lesions consisting of dense connective tissue and ≤5% viable cardiomyocytes. In two sections, there were viable Purkinje fibers seen in the areas between lesions and normal myocardium.

Clear MDE was visualized for Study 4 at 3 distinct locations along the RV septum. This was seen on both the 14 Day and 30 Day cMRI. On gross inspection, there were two spherical nodules within a larger lesion, firm to palpation. Pathology examination of these locations showed subendocardial fibrosis with associated adherent organizing thrombus.

For Study 5, a distinct area of MDE was observed in the RV apex as well as superficial MDE along the mid RV septal region. There was a corresponding focus of fibrotic-appearing tissue in the RV apex as well as a larger area of superficial scarring along the RV septum on gross inspection; these likely corresponded to the locations of the helix and ring respectively. Histopathologic examination showed dense areas of connective tissue with <5% residual cardiomyocytes in these samples.

Overall, the lesions associated with PEF delivery (dense fibrosis and minimal residual cardiomyocytes) were distinct in appearance from the features of lesions created by RFA, where there were irregular areas of necrotic cardiomyocytes interspersed with fibrosis.

### Discussion

This study demonstrated the feasibility of performing PEF ablation in ventricular tissue with a screw-in device using transvenous electroporation leads. With a single lead, lower energy PEF delivery resulted in mild bipolar voltage amplitude changes with transient unipolar ST elevations; no evidence of lesion creation was seen on cMRI or histopathology analysis. With higher energy deliveries enabled by a double electroporation lead configuration, durable bipolar voltage amplitude decreases developed post-PEF with corresponding pathologic lesions at end-study. No sustained ventricular arrhythmias were noted during PEF and survival period. These findings provide proof-of-concept evidence that PEF can be safely and effectively performed in ventricular myocardium targeted by active fixation.

### Active Fixation of Ventricular Myocardium for Pulsed Electric Field Delivery: Potential Benefits

Catheter ablation of VAs arising from the mid-myocardium remains a constant challenge for the invasive electrophysiologist. Strategies to address mid-myocardial substrate include: simultaneous RFA across opposing sides of the ventricular myocardium; half-normal saline administration (to create deeper lesions); RFA plus saline infusion via a needle catheter, and; retrograde coronary venous ethanol ablation. Of these, the needle ablation catheter is most similar to the approaches used in this study. However, an active fixation mechanism as used by the electroporation devices herein can offer improved positional stability in the targeted myocardium, which in turn can increase reliability of ablation and treatment effect assessment. Furthermore, a needle design may carry a greater risk of perforation, especially in the context of ventricular ectopy induced during PEF (as observed).

This property also couples well with PEF as an avenue for myocardial ablation. Numerous preclinical and clinical studies have demonstrated that PEF can be effectively employed for pulmonary vein (PV) isolation procedures without incurring the feared complications—PV stenosis, atrioesophageal fistula—typically associated with thermal-based ablation modalities. The optimal PEF parameters for ventricular ablation remain undefined at this nascent stage of development, which prompted our group to explore innovative techniques for expanding its utility. Fundamentally, the electric field vector plays an important role in defining the target lesion. PEF performed between two closely spaced electrodes can theoretically confine electroporation effects to a small location without affecting other regions of the ventricles. A PEF-capable screw-in electroporation device design therefore lends itself well to deep myocardial ectopic foci or substrate critical for maintaining reentrant ventricular tachycardia (VT).

### Demonstration of Reversible and Irreversible Electroporation Effects

With the above in mind, the first attempt was to use helix-to-ring PEF delivery with a single electroporation lead. Energy delivery was restricted because of limitations inherent to the electroporation lead. Even so, significant EGM changes were observed immediately post-PEF, most of which normalized within a few minutes. This may represent effects of reversible electroporation, which may be a useful feature to harness. For example, a reversible effect may be useful when ablation is delivered in regions where collateral damage is crucial (close to the conduction system to assess for heart block risk); additionally, it may be helpful to anticipate ablation effectiveness if the fixed location is in a location critical for initiating/sustaining VAs.

Placing two electroporation leads allows for increased energy delivery; consequently, more prominent and lasting electrical changes manifested, likely secondary to irreversible electroporation. This is further substantiated by observations of lesions compatible with irreversible electroporation effects. Interestingly, a difference was noted in the nature of lesions associated with the helix (deeper and more focal) and ring (superficial and more diffuse) from Study 5, which highlights the importance of the cathode/anode shape. Although true helix-to-ring irreversible electroporation was not demonstrated, this preclinical study provides compelling data that PEF delivery with a screw-in electroporation catheter design can be feasibly and safely performed in ventricular tissue. Optimization of an intentionally designed device with higher tolerance to the large current required can therefore be expected to yield evidence of irreversible electroporation.

### Nanosecond Pulsed Electric Field Delivery: Feasibility for Cardiac Ablations

Most electroporation generators in the preclinical and clinical arena deliver pulses in the microsecond and millisecond range. However, the inventors have found that nanosecond PEF can yield deeper and more uniform lesions in myocardial tissue compared to wider pulse width deliveries. Furthermore, shorter pulse widths may stimulate skeletal muscle to a lesser extent, which consequently lowers the risk of unwanted movement or barotrauma. The inventors demonstrated successful nanosecond PEF delivery to atrial and ventricular myocardium in several studies. Of note, the inventors have seen VT (probably automatic in etiology) and atrioventricular block with nanosecond PEF in the ventricular septum.

### Conclusions

Ventricular electroporation is feasible and safe with an active fixation device. Reversible changes were seen with lower energy PEF delivery, whereas durable lesions were created at higher energies. This study showed that low energy PEF with a single lead yielded transient electrogram changes and no histologic abnormalities at end-study, whereas high energy PEF with two leads led to persistent electrogram changes and permanent lesion formation. Lower energy deliveries with a single lead induced transient EGM changes consistent with reversible electroporation, whereas higher energy deliveries with two leads led to irreversible electroporation effects. This provides proof-of-concept feasibility for a PEF capable screw-in device to perform deep myocardial ablation. In addition, this study showed that an active fixation design can be utilized in targeting and ablating ventricular myocardial tissue using PEF ablation to treat ventricular arrhythmia by inducing permanent lesions. The greater stability from the screw-in design in combination with the exquisite myocardial sensitivity towards PEF delivery offer exciting promise for targeted and effective lesion creation in the ventricle. While much work remains to be done, the present study provides initial evidence for the concept's feasibility in the preclinical arena.

### New Electroporation Delivery Systems and Devices

Electroporation is a non-thermal mode of ablation consists of short high-energy pulses of DC ablation. It induces cell death by formation of nanopores within the membrane while preserving non-cellular tissue components. As it is low-energy, it is not associated with barotrauma and arcing, complications which prohibited widespread adoption of DC ablation. Multiple animal studies have been performed, as described above, showing the benefit of electroporation and relative safety. In addition, data suggest that compared with heat-based ablation approaches, IRE is more precise and results in clear demarcation lines between healthy tissue and ablated zone.

As described further below, this disclosure describes electroporation catheters with multiple different electroporation needle designs: helical, multi-helical, dual needle, multiple needles or a combined approach (figures). The needles would be plunge needles or screw in, effectively with only a small amount that would be exposed and inserted into the myocardium once the catheter is stabilized in position. These needles or helical needles would also have capability of placing electrodes onto the needles themselves to record intramyocardial unipolar and/or bipolar signals. The configuration will enable both unipolar and bipolar pulsed field ablation, in addition to traditional radiofrequency. These devices may also have multiple return electrodes along the shaft or the device or to a return surface patch or device placed in another cavity. In addition, electric bioimpedance tomography and a thermistor may be used for real-time assessment of temperature and control of the extent of ablation.

Such catheters are shown, for example in FIGS. 9-11, and 12-15. Referring first to FIGS. 9-11, the example catheter 200 includes a shaft 210 and a selectively deployable helical anchor 220 that can penetrate tissue when deployed to extend distally from the distal end of the shaft 210.

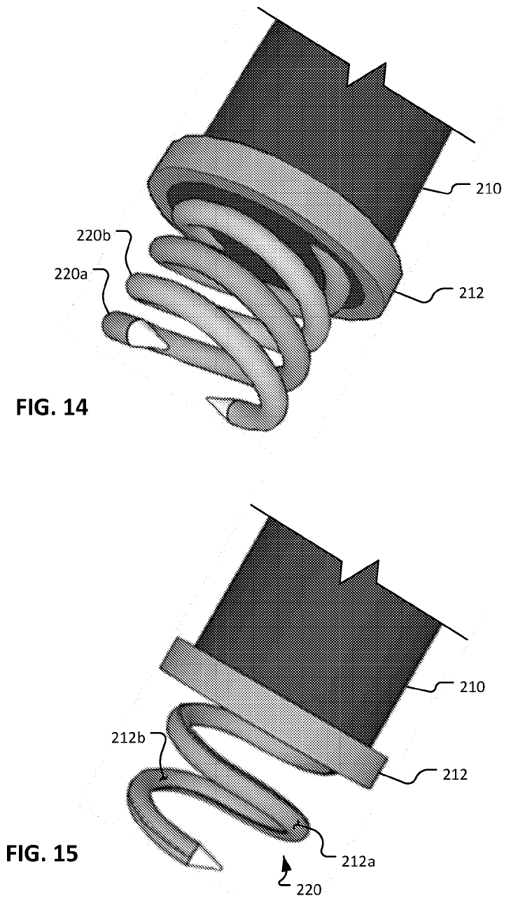

While the depicted embodiment of the catheter 200 shows a single helical anchor 220, in some embodiments two of the helical anchors 220a and 220b can be deployed from the single catheter 200, e.g., as depicted in FIG. 14. In some cases, the embodiment shown in FIG. 14 can utilize one of the two helical anchors 220a-b as an anode and the other of the two helical anchors 220a-b as a cathode to deliver electroporation energy to tissue in which the two helical anchors 220a-b have penetrated. The catheter shaft 210 can include one or more electrodes 212.

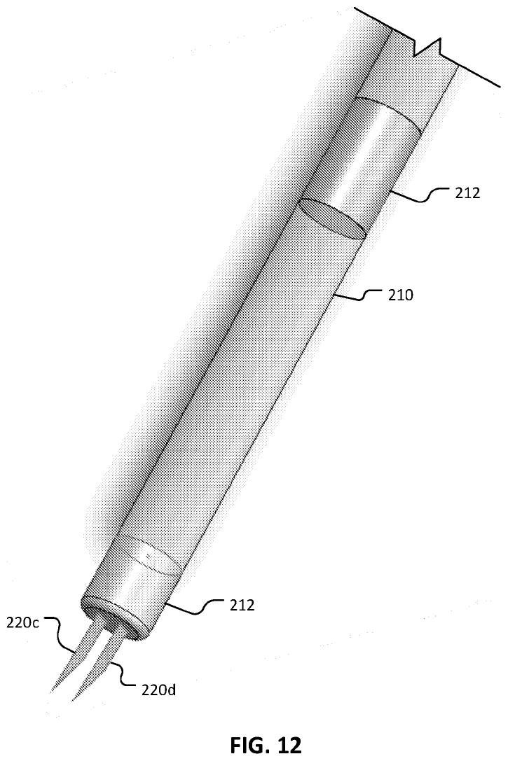

In some embodiments, instead of having one or two of the helical anchors 220, one or two straight needles that are selectively deployable can be substituted instead. One example embodiment of this configuration is depicted in FIG. 12. In some embodiments, the straight needles 220c and 220d are selectively deployable (e.g., distally extendable and proximally retractable) from the catheter shaft 210. In certain embodiments, one of the straight needles 220c-d can be solid while the other one of the straight needles 220c-d can be hollow (e.g., for drug delivery). In some embodiments, the hollow one of the straight needles 220c-d can include one or more side fenestrations through a wall of the needle (in addition to an opening at the distal-most tip end of the needle). In use, sometimes one of the straight needles 220c-d can be operated as an anode while the other one of the straight needles 220c-d can be operated as a cathode. Alternatively, in some embodiments both of the straight needles 220c-d can be operated as a cathode or an anode, and one or more electrodes 212 on the catheter shaft 210 can be operated as the anode or cathode (or a skin patch can be operated as the anode or cathode). Lastly, cryo may be used from a needle or distal cap on catheter to assist in tissue contact via the use of −20 degree Celsius to be delivered when in contact with the tissue. This will help ensure tissue contact during muscle contraction and delivery.

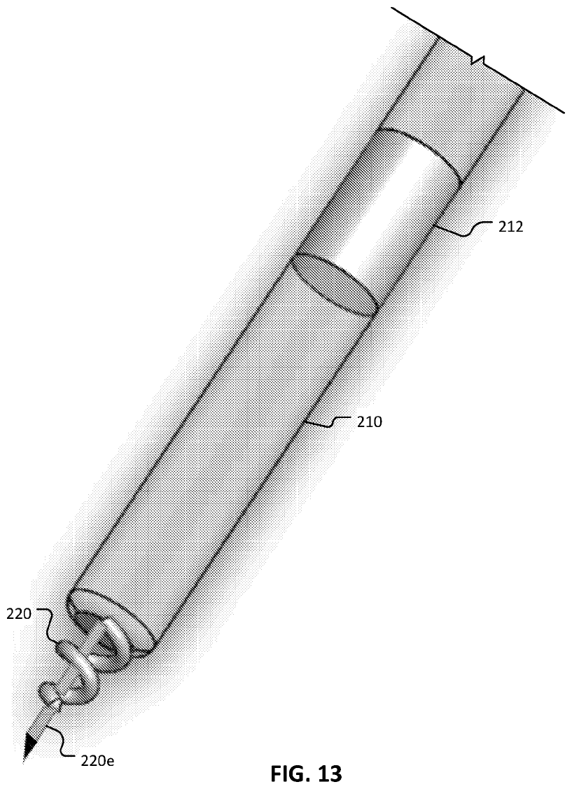

As shown in FIG. 13, in some embodiments, the catheter 200 can include both a selectively deployable helical anchor 220 and a selectively deployable straight needle 220e that is disposed within the interior defined by the helical anchor 220. The selectively deployable helical anchor 220 can be configured for various uses. In some embodiments, the selectively deployable helical anchor 220 is solely used for anchoring to tissue. In some embodiments, the selectively deployable helical anchor 220 is used for anchoring and as an electrode for delivering electroporation energy to the tissue in which it is penetrated. In some embodiments, the selectively deployable helical anchor 220 is or includes an electrode used for mapping/recording. In particular embodiments, the selectively deployable helical anchor 220 can be used to deliver cryogenic tissue ablation treatments.

In some embodiments, as shown in FIG. 15, the selectively deployable helical anchor 220 can include a first electrode 212a and a second electrode 212b that are separated by insulative surfaces on the selectively deployable helical anchor 220. The electrodes 212a-b can each extend along portions of, or the entirety of, the helical anchor 220 on opposite sides thereof (e.g., 1800 opposite of each other). In some cases, one of the two electrodes 212a-b is used as an anode and the other of the two electrodes 212a-b is used as a cathode to deliver electroporation energy to tissue in which the two helical anchors 220a-b have penetrated. The catheter shaft 210 can include one or more electrodes 212.

In some embodiments, one or more portions of the catheter shaft 210 are selectively steerable by a user of the catheter 200.

As described in the studies above, the deployable needle(s) or helical anchor 220 can be used to deliver and/or receive electroporation energy (as an anode and/or cathode) that is used to deliver electroporation to tissue such as, but not limited to, the interventricular septum 12.

In some embodiments, the catheter 200 (in any of the variations described herein) can include one or more electrodes 212 (FIG. 10) on the shaft 210 and/or the needle or helical anchor 220 (FIG. 11) with the capability to sense electrical activity in cardiac regions of interest, and or to act as a cathode. In addition, in some embodiments the catheter 200 (in any of the variations described herein) can have a location sensor to assist in anatomical mapping with contemporary 3D electroanatomical systems. Once the site of desired ablation is located, the needle or helical anchor 220 can be extended, and then intramyocardial mapping can be performed and electroporation delivered while maximizing stability. In some embodiments, the catheter 200 (in any of the variations described herein) can also optionally include a balloon 230 (FIG. 11) to provide additional stability, to allow for the application of atraumatic pressure against the tissue surface, and/or to act as a large return electrode. The balloon 230 may be filled with saline (isotonic, hypotonic, hypertonic solution).

In some embodiments, the catheter 200 (in any of the variations described herein) may also include one or more ports by which the catheter 200 has the ability to deliver fluid to the area of interest either through the needle, helix, or on the shaft or end of the catheter 200. The fluid may consist of saline, calcium, botulinum toxin, steroids, or any biological product.

In some embodiments, the catheter 200 (in any of the variations described herein) may include photo-biomodulation light delivery through the needle or the distal tip of the catheter 200. In some embodiments, the light wavelength can be in the spectrum of 300 nm to 900 nm. This light can be delivered in high energy levels to assist with changing the cellular impedance prior to ablation or can be used as the source of ablation. Light can also be delivered after ablation to assist with muscle contraction.

In some cases, irreversible electroporation can be delivered without the delivery of reversible electroporation. In some cases, the combination of electroporation and drug administration can reduce the concentration and/or amount of drug necessary to facilitate the same effects without electroporation. Such a decrease in amount and/or concentration of the drug administered can enhance the safety profile of the procedure. In some cases, the combination of electroporation and drug administration can reduce the energy required for electroporation. For example, drugs can be administered to specific tissues prior to electroporation. In some cases, the drug uptake can lower the threshold for cells to be ablated. Such a lowered threshold can enhance the safety and efficacy of ablation. In some cases, by lowering the threshold for electroporation, and/or ablation, the specific tissues can be more readily targeted, as the energy for electroporation would be sub-threshold when compared to surrounding tissue due to the differential of drug uptake.

In some cases, various methods can be used to increase time and/or surface area contact to allow for maximum cellular uptake of the drug. In some cases, such methods can alleviate or reduce the concern for drug washout through the systemic circulation from a beating heart. In some cases, a drug can be used to slow and/or stop the heart to allow for more time for drug activity. In some cases, the catheter assembly can include an adaptable catheter tip to enhance tissue-catheter contact. For example, the catheter tip can create a vacuum seal between the tissue and the catheter tip to allow for selective drug delivery and prevention of drug washout in the circulation. In some cases, the catheter tip can be deflectable to aid in positioning the catheter tip in proximity with the tissue. These can be used in conjunction and at the time of the percutaneous support to permit filling of the desired heart chamber with drug, expiration of specified amount of time, and then removal of the drug to reduce systemic toxicity.

In some cases, the systems and methods provided herein can provide reversible and transient termination of conduction in response to specific times when an arrhythmia takes place. Such a configuration can be an alternative form of defibrillation. For example, a drug delivery system can be attached to, or in communication with, the heart and can release a Purkinje specific drug and a low-level current (e.g., DC current) to permit Purkinje specific penetration of the therapeutic agent. In some cases, systemic effects can be reduced using a combination of electroporation and drug delivery. In some cases, the Purkinje specific drug can be reversible in the effect caused by the drug but allow for acute termination of the arrhythmia. In some cases, the system can include a mesh like portion with penetrating ports that are applied epicardially to the heart or a network applied along the endocardial surface with the capacity to deliver either a fluid or semi-fluid agent as well as a DC electric current.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described herein as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

1. An electroporation catheter device comprising:
a catheter shaft; and
a helical anchor or needle member that is selectively extendable from a distal end of the catheter shaft, wherein the helical anchor member is configured to deliver pulsed-electric field non-thermal electroporation ablation energy to tissue of a heart.

2. The catheter device of claim 1, further comprising a balloon member attached at a distal end portion of the catheter shaft.

3. The catheter device of claim 2, further comprising one or more electrodes on an outer surface of the balloon or in an interior of the balloon.

4. The catheter device of claim 1, further comprising a straight needle that selectively extendable from the distal end of the catheter shaft and within an interior region defined by the helical anchor member.

5. The catheter device of claim 1, further comprising one or more electrodes on the catheter shaft.

6. The catheter device of claim 1, further comprising a location sensor to assist in anatomical mapping.

7. The catheter device of claim 1, wherein the catheter shaft or the helical anchor member defines a port for delivering a fluid.

8. The catheter device of claim 1, wherein the catheter shaft or the helical anchor member is configured to deliver photo-biomodulation light.

9. A method for treating ventricular fibrillation of a heart, the method comprising:
using one or more of the catheter devices to deliver pulsed-electric field non-thermal electroporation ablation energy to one or more target locations of the heart,
wherein said one or more of the catheter devices are electroporation catheter devices comprising:
a catheter shaft; and
a helical anchor or needle member that is selectively extendable from a distal end of the catheter shaft, wherein the helical anchor member is configured to deliver pulsed-electric field non-thermal electroporation ablation energy to tissue of a heart.

10. The method of claim 9, wherein the one or more target locations of the heart includes an endocardial space of the heart, the mid-myocardium of the heart, and an epicardial space of the heart.

11. The method of claim 9, wherein the one or more target locations of the heart includes a ventricular septum of the heart.

12. The method of claim 11, further comprising using two of the catheter devices and penetrating the ventricular septum with two of the helical anchor or needle members on a same side of the ventricular septum.

13. The method of claim 12, wherein a first one of the helical anchor members functions as an anode and a second one of the helical anchor members functions as a cathode.

14. The method of claim 11, further comprising using two of the catheter devices and penetrating the ventricular septum with two of the helical anchor members on opposite sides of the ventricular septum.

15. The method of claim 14, wherein a first one of the helical anchor members functions as an anode and a second one of the helical anchor members functions as a cathode.

16. The method of claim 9, further comprising monitoring tissue impedance prior to and during the delivery of the pulsed-electric field non-thermal electroporation ablation energy.
